(19) United States
(12) Patent Application Publication
(10) Pub. No.: US 2023/0346993 A1
(43) Pub. Date: Nov. 2, 2023

(54) ULTRAVIOLET STERILIZATION FOR MINIMALLY INVASIVE SYSTEMS

(71) Applicant: Cercacor Laboratories, Inc., Irvine, CA (US)

(72) Inventors: Massi Joe E. Kiani, Laguna Niguel, CA (US); Kevin Hughes Pauley, Lake Forest, CA (US)

(21) Appl. No.: 18/139,757

(22) Filed: Apr. 26, 2023

Related U.S. Application Data
(60) Provisional application No. 63/335,680, filed on Apr. 27, 2022.

(51) Int. Cl. A61L 2/00

(52) U.S. Cl. CPC A61L 2/0047

(57) ABSTRACT

Systems, methods, and apparatuses for enabling the emission and reflection of UV-C light to sterilize a patient’s skin. A UV chip can be configured to emit the UV-C light. A UV reflector can receive the UV-C light and divert it toward the patient’s skin. The UV-C light can be directed to shine through a UV window to sterilize the patient’s skin.

### CROSS REFERENCE TO RELATED APPLICATIONS

This application claims benefit of priority under 35 U.S.C. § 119(e) to U.S. Provisional Application No. 63/335,680, filed on Apr. 27, 2022, entitled “ULTRAVIOLET STERILIZATION FOR MINIMALLY INVASIVE SYSTEMS,” which is hereby incorporated by reference in its entirety.

### FIELD OF THE DISCLOSURE

The general field of this disclosure is ultraviolet sterilization, specifically for use in glucose sensing and disease management systems.

### BACKGROUND

An area of concern in minimally invasive or infusion treatment related devices is the ability for the device to maintain sterility during use. As devices become longer lasting and are designed to be worn for more extended periods of time on the body, the chance of compromising sterility increases and in turn, so does an increased opportunity for infection at an insertion site of the device. In some situations, the insertion can be viewed as an open wound and therefore also elicits cellular damage concern of small cells. So, special attention may be provided so as not to pipe into and cause additional tissue damage. Accordingly, it is desirable to ensure sterility of both the inserted or implanted portion of a minimally invasive device and the site of insertion or contact to reduce risk of infection.

### SUMMARY

Various aspects of systems, methods, and devices within the scope of the appended claims each have several aspects, no single one of which is solely responsible for the desirable attributes described herein. Without limiting the scope of the appended claims, some prominent features are described herein.

Short wavelength ultraviolet light, such as Ultraviolet-C or UV-C (or ultraviolet light having a wavelength in a range of between 100 and 280 nm) can be used for disinfection of surfaces, including human skin. UV-C light has a short penetration distance in skin. For example, the range of UV dispersion through skin can be less than 5 microns with an average circular area of less than 0.01 mm² for a point source. The very short penetration distance means mammalian cells, which are greater than 10 microns in diameter, do not undergo DNA destruction and remain intact when exposed to UV-C light. Due to the relatively short penetration distance, to be effective as a method of disinfection of the skin, attention to the details of light placement is helpful.

An ultraviolet sterilization device can be used to sterilize the underside of a glucose sensing or disease management system. Additionally, or alternatively, sterilization can occur around the point of insertion. The ultraviolet sterilization device can include a UV light emitter configured to emit UV light, a light pipe configured to direct light from the UV light emitter, a UV reflector to receive the emitted UV light and divert it toward the patient’s skin, and a UV window positioned between the UV reflector and the patient’s skin such that the diverted UV light is transmitted through the UV window to sterilize the patient’s skin.

In some aspects, the techniques described herein relate to a minimally invasive implant worn flush against a patient’s skin including: an implantable component configured to at least partially implant into the patient’s skin for a period of time of use of the minimally invasive implant; a UV light emitter configured to emit UV-C light; at least one UV reflector configured to receive light from the UV light emitter, the at least one UV reflector including: an interior cavity including: an implant receiving portion running lengthwise along the at least one UV reflector and configured to receive at least some of the implantable component; and an angled surface configured to receive UV-C light from at least one light channel perpendicular to a centerline of the interior cavity and to direct the received UV-C light towards an exit opening of the interior cavity such that the implantable component is sterilized by the UV-C light, wherein the at least one light channel is configured to receive the UV-C light from the UV light emitter; and a UV window positioned between the at least one UV reflector and the patient’s skin such that diverted UV-C light shines through the UV window to sterilize the patient’s skin, the UV window including an opening configured to allow the implantable component to at least partially pass through the UV window to implant in the patient’s skin.

In some aspects, the techniques described herein relate to a minimally invasive implant, wherein the interior cavity is reflective to UV-C light and able to receive UV-C light to reflect the UV-C light within the at least one UV reflector towards a proximal exit opening of the UV reflector.

In some aspects, the techniques described herein relate to a minimally invasive implant, wherein the UV light emitter is connected to the at least one UV reflector via at least one light pipe.

In some aspects, the techniques described herein relate to a minimally invasive implant, wherein the UV light emitter includes a first UV chip configured to emit light to the UV reflector and a second UV chip configured to emit light to a second UV reflector.

In some aspects, the techniques described herein relate to a minimally invasive implant, wherein the UV reflector receives a first needle and an analyte sensor, and the second UV reflector receives a second needle and a cannula.

In some aspects, the techniques described herein relate to a minimally invasive implant, wherein the angled surface includes an angle of approximately 47.5 degrees.

In some aspects, the techniques described herein relate to a minimally invasive implant, wherein the at least one UV reflector includes a diameter of approximately 3.50 mm and a height of approximately 3 mm.

In some aspects, the techniques described herein relate to a minimally invasive implant, wherein the at least one UV reflector further includes a tube extending towards an exit opening of the cavity.

In some aspects, the techniques described herein relate to a minimally invasive implant, wherein the at least one UV reflector couples to a top shell of a disease management device.

In some aspects, the techniques described herein relate to a minimally invasive implant, wherein the UV window couples to a lower shell of a disease management device.

In some aspects, the techniques described herein relate to a minimally invasive implant, wherein the UV-C light undergoes a filtration process prior to sterilizing the patient’s skin.

In some aspects, the techniques described herein relate to a minimally invasive implant, wherein the UV light emitter is further configured to emit light periodically.

In some aspects, the techniques described herein relate to a minimally invasive implant, wherein the UV light emitter is further configured to provide a 1 second exposure over 3600 seconds.

In some aspects, the techniques described herein relate to a minimally invasive implant, wherein a light channel is positioned to connect an outer surface of the at least one UV reflector to the angled portion of the at least one UV reflector.

In some aspects, the techniques described herein relate to a method to sterilize minimally invasive device and insertion site of a patient, the method including: emitting UV-C light from a UV emitter periodically to be received by at least one UV reflector within a disease management system; and reflecting UV-C light within the at least one UV reflector to sterilize at least a portion of the disease management system and insertion site of the patient at a predetermined frequency.

In some aspects, the techniques described herein relate to a method, wherein the predetermined frequency is based on an area of the insertion site.

In some aspects, the techniques described herein relate to a method, wherein the predetermined frequency is based on a degree of risk for infection at the insertion site.

In some aspects, the techniques described herein relate to a method to sterilize an insertion site of a patient, the method including: inserting a needle of a disease management system into the insertion site of the patient; collimating UV light to enter at least one UV reflector located within an UV sterilization device; reflecting UV light to direct the UV light inward toward a center axis of the at least one UV reflector, wherein the needle is positioned within with the center axis of the at least one UV reflector; diffusing the UV light through a UV window upon the UV light exiting the at least one UV reflector, wherein the UV window is configured to allow the needle to pass through the UV window to the insertion site of the patient; and sterilizing the insertion site of the patient.

In some aspects, the techniques described herein relate to a method further including conditioning a light source prior to collimating UV light.

In some aspects, the techniques described herein relate to a method wherein conditioning the light source includes bandpass filtering emitted UV light.

In some aspects, the techniques described herein relate to a method, wherein the UV window is flush against the patient.

In some aspects, the techniques described herein relate to a method further including re-sterilizing the insertion site of the patient based on a spot size of the UV light.

### BRIEF DESCRIPTION OF THE DRAWINGS

Throughout the drawings, reference numbers may be re-used to indicate correspondence between referenced elements. The drawings are provided to illustrate example configurations described herein and are not intended to limit the scope of the disclosure.

FIG. 1 illustrates an example disease management system that may be part of a disease management environment or used as an interleaved device.

FIG. 2 illustrates an example implementation of a disease management system.

FIG. 3 illustrates a perspective view of an example infusion treatment device.

FIG. 4A is a cross section view of an analyte sensor and needle of an infusion treatment device.

FIG. 4B is a cross section view of a cannula and needle of an infusion treatment device.

FIG. 5A illustrates a perspective view of an example top shell.

FIG. 5B illustrates a side view of an example top shell.

FIG. 6A illustrates a perspective view of an example UV Reflector.

FIG. 6B illustrates a top view of an example UV Reflector.

FIG. 6C illustrates a side view of an example UV Reflector.

FIG. 6D illustrates a cross section view of an example UV Reflector.

FIGS. 7A-7B illustrates an example optical system.

FIGS. 8A-8B illustrates a perspective view of an example UV window.

### DETAILED DESCRIPTION

Although certain preferred aspects and examples are disclosed below, inventive subject matter extends beyond the specifically disclosed examples to other alternative aspects and/or uses and to modifications and equivalents thereof. Thus, the scope of the claims that may arise here from is not limited by any of the particular aspects described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various aspects, certain aspects and advantages of these aspects are described. Not necessarily all such aspects or advantages are achieved by any particular aspect. Thus, for example, various aspects may be carried out in a manner that achieves or optimizes one advantage or group of advantages as taught herein without necessarily achieving other aspects or advantages as may also be taught or suggested herein.

Systems and methods described herein relate to sterilization of parts associated with a minimally invasive implant and/or skin near an implant or insertion site associated with a minimally invasive implant. Systems and methods may include an Ultraviolet (UV) light source configured to disinfect portions of the minimally invasive implant and/or skin during use of the minimally invasive implant. A minimally invasive implant can include separate and/or combination insulin pump and analyte sensor or monitor. However, other applications of the UV sterilization system are also possible. The sterilization system and methods described herein can be applicable to any device needing at least some level of sterilization or disinfection before or during use.

### A. Glucose Monitor and Insulin Pump

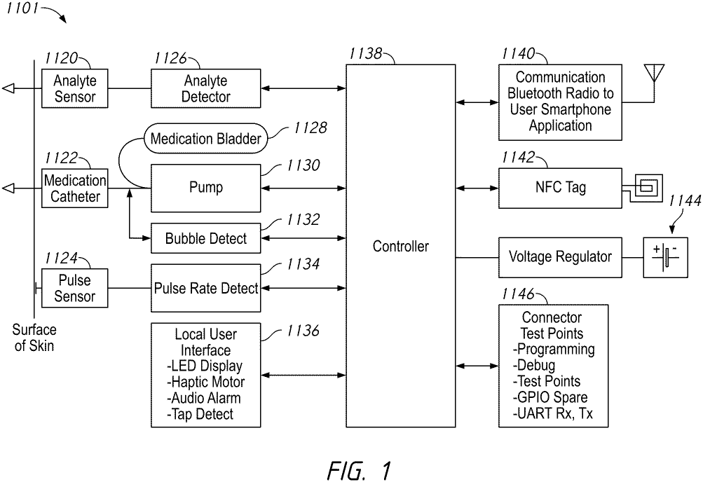

FIG. 1 shows a block diagram of an example disease management system 1101. A disease management system may include the sensor, the medication pump, the sensor and the medication pump combined, or any of the foregoing with further physiological sensors. The disease management system 1101 may be part of a disease management environment. A disease management system 1101 may be configured to measure one or more physiological parameters of a patient (such as pulse, skin temperature, or other values), measure one or more analytes present in the blood of a patient (such as glucose, lipids, or other analyte) and administer medication (such as insulin, glucagon, or other medication). A disease management system 1101 may be configured to communicate with one or more hardware processors that may be external to the disease management system 1101, such as a cloud-based processor or user device. A disease management system 1101 may include an NFC tag to support authentication and pairing with a user device (for example, smart phone or smart watch), Bluetooth communication with additional disease management systems or devices, and Bluetooth communication with a paired user device running an associated control application. To support ease of use and safe interaction with the patient, the system may incorporate user input through a tap-detecting accelerometer and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 1, a controller 1138 of the disease management system 1101 may be configured to communicate and control one or more components of the disease management system 1101. The controller 1138 may include one or more hardware processors, such as a printed circuit board (PCB) or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer medication, such as insulin, by actuation of an actuated pump. The controller 1138 may record device activity and transfer the recorded data to non-volatile secure memory space. At the end of the life of a device or system, the controller can be configured to lock operation, and create a data recovery module to permit authenticated access to the recorded data if needed.

A disease management system 1101 may include an analyte sensor 1120. The analyte sensor 1120 may be configured to detect analytes in the patient’s blood. For example, an analyte sensor 1120 can include a glucose sensing probe configured to pierce the surface of the skin 1121. A disease management system 1101 may include a plurality of analyte sensors 1120 to detect one or more analytes. An analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

In addition to the analyze sensor or alternatively to the analyte sensor, the disease management system 1101 may include a medication catheter 1122. The medication catheter 1122 may be configured to administer medication, including, but not limited to insulin, to the patient. The medication catheter 1122 may receive medication from a medication bladder 1128 configured to contain medication to be administered. The medication bladder 1128 may be configured to contain medication for a prolonged period, such as 1 day, 3 days, 6 days, or more. The medication bladder 1128 may be configured to contain certain medication types, such as insulin. A disease management system 1101 may include a plurality of medication bladders 1128 for one or more reservoirs of the same or different medications. A disease management system 1101 may be configured to mix medications from medication bladders 1128 prior to administration to the patient. A pump 1130 may be configured to cause medication to be administered from the bladder 1128 to the patient through the insulin catheter 1122. A pump 1130 may include, but is not limited to, a pump such as described herein.

A disease management system 1101 may optionally include a physiological sensor 1124. The physiological sensor 1124 may include a pulse rate sensor, temperature sensor, pulse oximeter, the like or a combination thereof. A disease management system 1101 may be configured to include a plurality of physiological sensors. The physiological sensor 1124 may be configured to communicate with a physiological detector 1134. The physiological detector 1134 may be configured to receive signals of the physiological sensor 1124. The physiological detector 1134 may be configured to measure or determine and communicate a physiological value from the signal. The physiological detector 1134 may be configured to communicate with the controller 1138. For example, the physiological detector 1134 may be configured to, for example, send measured physiological values to the controller 1138 and receive control signals from the controller.

A disease management system 1101 may include one or more local user interfacing components 1136. For example, a local user interfacing component 1136 may include, but is not limited to one or more optical displays, haptic motors, audio speakers, and user input detectors. An optical display may include an LED light configured to display a plurality of colors. An optical display may include a digital display of information associated with the disease management system 1101, including, but not limited to, device status, medication status, patient status, measured analyte or physiological values, the like or a combination thereof. A user input detector may include an inertial measurement unit, tap detector, touch display, or other component configured to accept and receive user input. Audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include one or more communication components 1140. A communication component 1140 can include but is not limited to one or more radios configured to emit Bluetooth, cellular, Wi-Fi, or other wireless signals. A communication component 1140 can include a port for a wired connection. Additionally, a disease management system 1101 may include an NFC tag 1142 to facilitate in communicating with one or more hardware processors. The one or more communication components 1140 and NFC tag 1142 may be configured to communicate with the controller 1138 in order to send and/or receive information associated with the disease management system 1101. For example, a controller 1138 may communicate medication information and measured values through the one or more communication components 1140 to an external device. Additionally, the controller 1138 may receive instructions associated with measurement sampling rates, medication delivery, or other information associated with operation of the management system 1101 through the one or more communication components 1140 from one or more external devices.

A disease management system 1101 may include one or more power components 1144. The power components may include but are not limited to one or more batteries and power management components, such as a voltage regulator. Power from the one or more power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake-up mode. The wake-up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculation, are performed and data is communicated to an external device and/or the cloud. In another example, a disease management system 1101 may include a pump mode where the system is configured to enter a higher power mode where the pump draws power to deliver medication to the patient.

A disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, the like or a combination thereof.

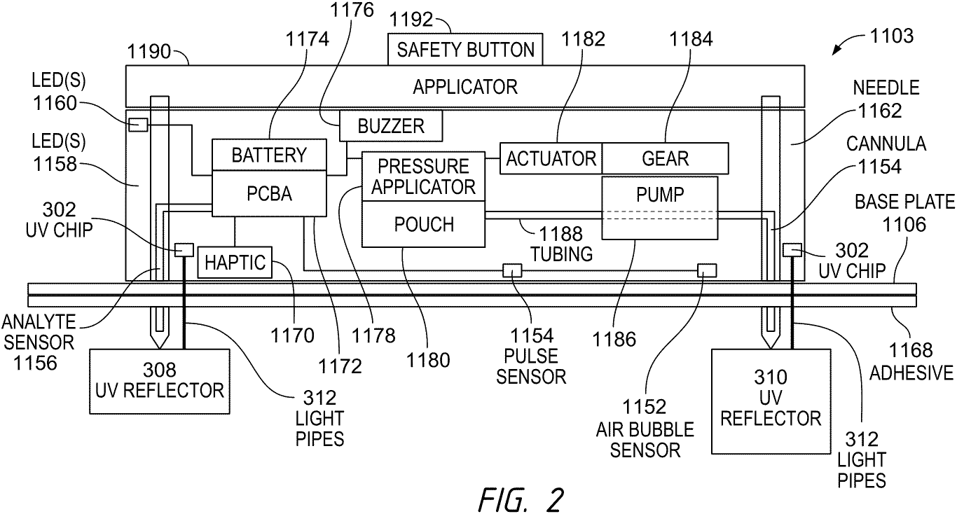

FIG. 2 illustrates an example implementation of a disease management system 1103 and applicator 1190 for applying a disease management system 1103 to a patient. Disease management system 1103 can include any one or more of the features discussed above with respect to the disease management system 1101 in addition to the features described below. In the illustrated example, an applicator 1190 may be configured to mate with the disease management system 1103. An applicator 1190 may include a safety button 1192 for release or other interaction with the applicator 1190. In the illustrated example, a disease management system 1103 may include one or more LEDs 1160 that may be configured to output information using one or more of color, frequency, and length of display. The disease management system 1103 may include a buzzer 1176, haptic actuator 1170, or other feedback mechanism, such as a speaker to output information to the patient, such as an alarm. A disease management system 1103 may include a battery 1174, controller 1172. A disease management system 1103 may include aspects of a medication administration system, such as a bladder 1180, a bladder pressure applicator 1178 to provide pressure on the bladder (such as a component of a pump), actuator 1182, pump gears 1184, and a pump 1186. A disease management system 1103 may include one or more needles 1158 that may include one or more analyte sensors (such as a glucose sensor) 1156. A disease management system 1103 may include one or more needles 1162 that may include one or more cannulas 1164 configured to administer medication to the patient. A disease management system 1103 may include an air bubble sensor 1152 configured to detect the presence of air bubbles in the medication prior to delivery to the patient. A disease management system 1103 may include one or more physiological sensors 1154, such as a non-invasive physiological sensor including but not limited to a pulse sensor. The disease management system 1103 may include a base plate 1106 and an adhesive layer 1168 below the base plate 1106 to provide adhesion of the disease management system 1103 to the patient’s skin. As described below, a housing of the disease management system 1103 may consist of a combination of flexible and rigid material so as to both provide support for the components of the disease management system 1103 and allow conforming, at least in part, of the disease management system 1103 to the skin of the patient.

The adhesive layer 1168 may be configured to provide adhesion for a prolonged period. For example, the adhesive layer 1168 may be configured to adhere the disease management system 1103 to the skin of a patient for a period of 1 day, 3 days, 6 days, or more or fewer days or hours. The adhesive layer may be configured to have an adhesive force sufficient to prevent accidental removal or movement of the disease management system 1103 during the intended period of use of the disease management system 1103. The adhesive layer 1168 may be a single layer of adhesive across at least a portion of a surface the disease management system 1103 that is configured to interface with the patient. The adhesive layer 1168 may include a plurality of adhesive areas on a surface of the disease management system 1103 that is configured to interface with the patient. The adhesive layer 1168 may be configured to be breathable, adhere to the patient’s skin after wetting by humidity or liquids such as tap water, saltwater, and chlorinated water. A thickness of the adhesive may be, for example, in a range of 0.1 to 0.5 mm or in a range of more or less thickness.

A needle 1158, 1162 may be inserted at different depths based on a patient age, weight, or other parameter. For example, a depth of insertion of a medication cannula may be approximately 3 mm for 7- to 12-year-olds. In another example, a depth of insertion of a medication cannula may be approximately 4 mm for 13-year-olds and older. In another example, a depth of insertion of a medication needle may be approximately 4 to 4.5 mm for 7-to 12-year-olds. In another example, a depth of insertion of a medication needle may be approximately 5 to 5.5 mm for 13-year-olds and older. In another example, a depth of insertion of an analyte sensor may be approximately 3 mm for 7- to 12-year-olds. In another example, a depth of insertion of an analyte sensor may be approximately 4 mm for 13-year-olds and older. In another example, a depth of insertion for a needle associated with an analyte sensor may be approximately 4 to 4.5 mm for 7- to 12-year-olds. In another example, a depth of insertion for a needle associated with an analyte sensor may be approximately 5 to 5.5 mm for 13-year-olds and older. However, other values or ranges for any of the inserted components are also possible.

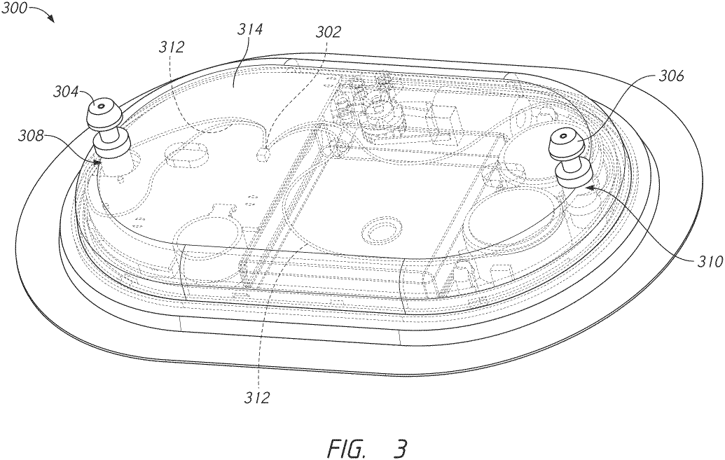

At least one UV reflector 308, 310, such as shown in FIG. 3, may be located at or near each insertion site where the needle 1158, 1162 may be inserted into the patient. The UV reflector(s) 308, 310 may receive UV light from a UV chip 302 via a light pipe 312, shown in FIG. 3. Further details of example UV reflector(s) 308, 310 are discussed herein.

An analyte sensor 1156, such as illustrated in FIG. 2, can include a micro-electrochemical cell configured to at least partially implantable into the tissue of the patient. The micro-electrochemical cell may include one or more sensor components enclosed at least in part in a permeable cell. The permeable cell may include one or more permeable portions configured to allow passage of analyte containing fluid from the surrounding tissue of the patient to a portion of the permeable cell containing the one or more sensor components. The one or more sensor components may be configured to measure at least one analyte, such as glucose or other analyte present at the tissue site of the patient.

### B. Infusion Treatment Related Device Overview

FIG. 3 illustrates an example minimally invasive implantable device that may use sterilization systems and methods such as described herein. An implantable device may include a disease management system or device 300 that may include a combined glucose monitor and insulin pump, referenced in FIG. 1 and FIG. 2, and described in U.S. Pat. Pub. No. 2021/0236729 filed Jan. 28, 2021 and titled “REDUNDANT STAGGERED GLUCOSE SENSOR DISEASE MANAGEMENT SYSTEM”, the entire contents of which are hereby incorporated by reference in its entirety. As illustrated in FIG. 3, a disease management system or device 300 can include a printed circuit board (PCB) 314, an analyte sensor and needle 304, and a cannula and needle 306. The disease management system 300 may also include a UV 222 chip 302 and light pipes 312. The UV chip 302 can be a UV light emitter. The system 300 may include more than one UV chip 302. This would provide a separate light source for both analyte sensor and needle 304 and the cannula and needle 306. Each UV chip 302 may include light pipes 312 directed to their respective UV reflectors 308, 310. For example, where a disease management system is modular, each modular section having components associated with insertion into the skin of the patient may include its own UV chip 302. Thus, sterilization of different components of the system may be accomplished separately if needed, such as when a single module is replaced and a second module remains in place (for example, an insulin pump module is replaced and an analyte sensor module remains implanted into the user).

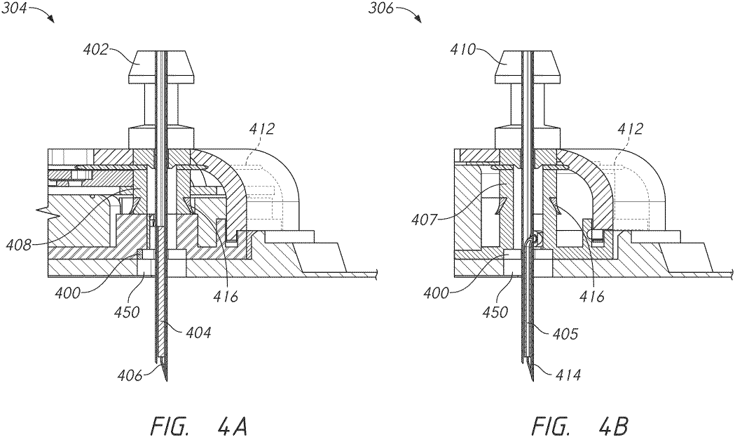

The UV chip 302 may be mounted on the PCBA 314. The UV chip 302 may emit UV-C light and may be connected to UV reflectors 308, 310 via light pipes 312. The UV-C light may originate at the UV chip 302. In some embodiments, there may be localization of the UV-C light at the UV chip 302 prior to the UV-C light transmitting through the light pipes 312. In some embodiments, the UV reflectors 308, 310 may ultimately receive the UV-C light. The light pipes 312 may be solarization-resistant optical fiber. The light pipes 312 may guide the UV-C light from the UV chip 302 to the area of the system 300 including the analyte sensor and/or associated needle 304 and cannula and/or associated needle 306 with minimal loss/dispersion of light. This may allow for improved sterilization of the patient’s skin. The analyte sensor and needle 304 and cannula and needle 306 may each be connected to a UV reflector 308, 310 (see, for example, FIGS. 4A-4B) which diverts the light toward the patient’s skin.

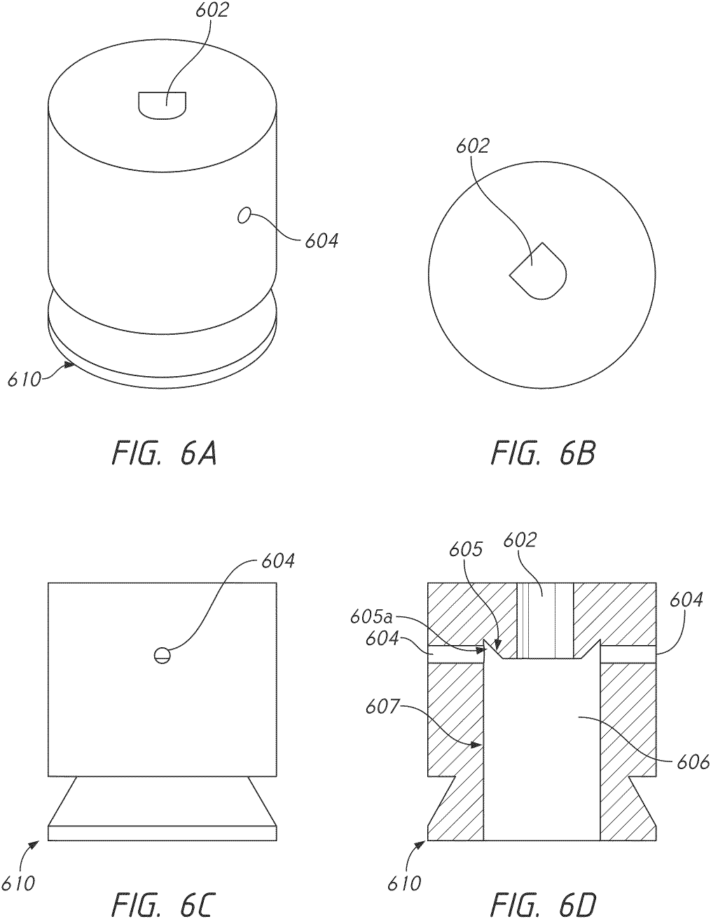

The system 300 may contain the analyte sensor and needle 304, the cannula and needle 306, or both. For example, the system 300 may contain a continuous glucose monitor (CGM) and/or may contain an insulin pump. The UV chip 302 and continuous glucose monitor may work together or dynamically cooperate with each other through the use of the system 300. For example, the UV chip 302 may emit UV-C light more than once depending on the use of the continuous glucose monitor or the treatment plan that incorporate the CGM. The needles may be used to implant the analyte sensor and/or cannula into a patient’s skin. Because incisions in the patient’s skin may be areas more prone to infection, the UV-C light may be concentrated at these device points of entry. The UV reflectors 308, 310 may therefore redirect the UV-C light toward the patient’s skin. Further description of the UV reflectors 308, 310 is discussed in reference to FIGS. 6A-D.

The UV chip 302 may emit light, such as UV-C light to sterilize various potential points of infection. In general, UV-C light exists in a wavelength range of 200 nm to 300 nm, which allows the light to be used as a sterilization technique. This is beneficial in sterilizing the insertion site. At these wavelengths, proteins absorb the UV-C light which may lead to rupture of cell walls of microorganisms and hinders the ability of the cells to replicate, effecting sterilization of the site. The UV chip 302 may emit UV-C light to sterilize the analyte sensor and needle 304 and/or the cannula and needle 306 prior to insertion, the insertion site before and/or during insertion of the analyte sensor and needle 304 and the cannula and needle 306, and/or the insertion site continuously or regularly after insertion. By sterilizing a potential point of infection prior to insertion, during insertion, and/or continuously or regularly after insertion, the risk for infection may be minimized. Furthermore, the frequency of the UV-C light exposure can allow for a low dose of the UV-C light to be used. The frequency of the UV-C light exposure may be a predetermined frequency. This minimizes the risks associated with UV-C light exposure to patients.

### C. Example UV Sterilization System

As illustrated in FIG. 4A and FIG. 4B, the system 300 may include a plurality of implantable components, such as some combination of needles, a glucose probe 404, and a cannula 405. A first needle 406 may be used to implant the glucose probe whereas a second needle 414 may be used to implant the cannula 405. In some embodiments, the first needle 406 may be implanted prior to or after the second needle 414. In some embodiments, the first needle 406 and the second needle 414 may be implanted at the same time. In other embodiments, the second needle 414 may be implanted prior to the first needle 406. The first needle 406 may be a hollow needle such that the glucose probe 404 fits within the hollow needle. The second needle 414 may be a solid needle capable of fitting into the middle of the cannula 405 which is a hollow tube. In some implementations, the second needle 414 may also be a needle with a hollow center and the cannula 405 may have a smaller diameter than the hollow center of the needle, which allows the cannula 405 to fit within the needle. At least a portion of the first needle 406 and the second needle 414 may insert into the insertion site. The portion of the first needle 406 that inserts into the insertion site may be more or less than the portion of the second needle that inserts into the insertion site.

The analyte sensor and needle 304 may include a needle holder 402, a glucose probe 404, a needle 406, a UV reflector 408, and UV windows 400, 450, shown in FIGS. 4A-4B.

The needle holders 402, 410 may hold the needles 406, 414 in place for greater stability when implanting the probe and cannula into the patient. The needle holders 402, 410 may or may not be removed from the device once the device is implanted into the patient’s skin. Removal may minimize the size of the insertion site and improve the accuracy of implanting the probe and cannula into the intended insertion site. Additionally, this may reduce pain or uncomfortableness felt by the patient during wear.

Implantable components may be received into a UV reflector 407, 408. A length and/or shape of the UV reflector 407, 498 may be different based on the application. The needles 406, 414 may pass through the center of the UV reflectors 407, 408. This can help ensure that the implantable components and/or insertion site are at least partially sterilized. The light path may be directed in a way in which may ensure sterilization of the skin at the points of insertion and/or the implanted parts of the implantable components are sterilized.

Light from light pipes 412 may be directed by the UV reflector(s) to sterilize the skin at or near the point of insertion into the skin. The light may be collimated in order for the light to enter the UV reflectors 407, 408. The collimation of light allows the intensity of the light to be regulated and controlled throughout the path of the light and the sterilization process. For example, the intensity of the light may vary depending on size of the insertion site. Similarly, the intensity of the light may vary depending on the frequency at which the insertion site is sterilized. Upon collimation and entrance of the light into the UV reflectors 407, 408, the light may be reflected to redirect the light inwards for the light to continue its path to the insertion site.

The light may undergo a filtration process. Such filtration may include physical bandpass filtering using diffraction grating or a technique of the like. This may allow only a certain spectrum of light, or a preferred spectrum of light sterilize the insertion site.

Upon exiting the UV reflectors, the light may be diffused. Diffusion of the light may allow for a larger area of the site of insertion to be sterilized. In this manner, this can help to ensure that the area that is sterilized. In some embodiments, the degree of sterilization may decrease as the light comes in contact with the insertion site further away from the exit point of the UV reflector. In some embodiments, the degree of sterilization may be approximately equal across the entirety of the insertion site even with the diffusion of light.

The UV reflector may be partially or entirely surrounded by a skirt 416 to seal in the UV-C light. The skirt 416 may be made of a silicone material. This may help to ensure the insertion site is sterilized. Alternatively, or in addition, the skirt 416 may be triangular in shape and seal UV light and water. Similarly, the needles 406, 414 may be encircled by the UV windows 400, 450 (see for example, FIGS. 4A-4B) through which the UV-C light may pass. The UV windows 400, 450 may lie approximately flush against the patient’s skin to maximize surface area exposed to the UV-C rays. This may further help to ensure the insertion site is sterilized. The skirt 416 may also act as a water seal such that patient bodily fluids cannot enter the device. The skirt 416 can be composed of other material suitable for forming tight seals.

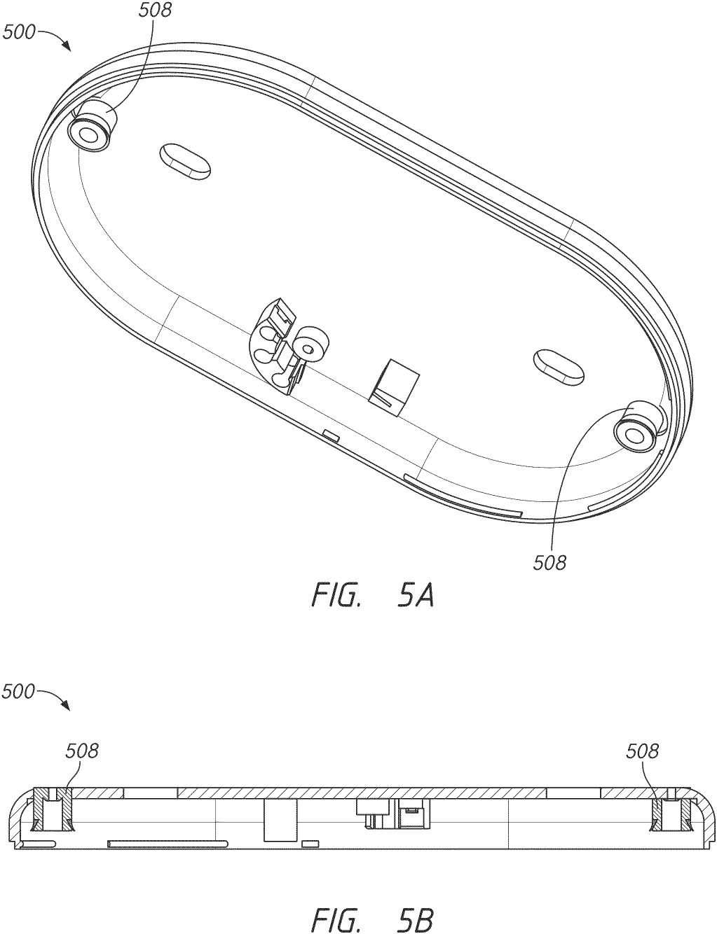

FIGS. 5A-5B illustrates a top shell 500. The top shell 500 may include at least one crevice. As mentioned earlier, the UV reflectors 508 may be press fit, glued, or generally coupled with the corresponding crevices in the top shell to reduce the risk of errors caused by thermal expansion during device operation. The top shell 500 may be configured to cover other components of the system 300. In this manner, the UV reflectors 508, when coupled with the corresponding crevices in the top shell 500, may align with the UV windows and the point of insertion to allow for the light received by the UV reflectors 508 to be directed to the point of insertion to sterilize the skin. Specifically, the UV reflectors 508 may be coupled with the top shell 500, rather than other components of the system 300, to ensure sufficient acceptance of the UV-C light by the UV reflectors 508. This coupling may further ensure a greater amount of light to be received than otherwise may be received. This may help to ensure the insertion site is sterilized. The top shell 500 can be a thickness in a range of approximately 0.5 mm to 5 mm, or a value less than or greater than that range. For example, a top shell 500 can be approximately 1 mm thick. The thickness of the top shell 500 may be proportional to the height of the UV reflector 508. The thickness of the top shell 500 may be longer in length compared to the height of the UV reflector 508 to control or regulate the diffusion of the light.

FIGS. 6A-6D illustrates an example UV reflector 608. The UV reflector 608 may be roughly cylindrical in shape and have a guide channel 602 running through its center lengthwise for a needle or other implantable device or portion thereof. The UV reflector 608 may include an interior cavity 606 configured to receive the needle or other implantable device. Reflective coating in the interior cavity 606 may allow UV light to interact with the received needle or other implantable device such that sterilization can occur. Additionally, the shape of the UV reflector 608 may be configured to direct light down the centerline or shaft of the UV reflector 608 such that a length of the needle or other implantable device within the cavity 606 is sterilized. Additionally, the light may be reflected towards an exit opening at a proximal end 610 of the UV reflector such that light may exit the exit opening and travel towards the insertion site of the needle or implantable device. Thus, the insertion site may additionally be exposed to UV light such that sterilization can occur. Further optics may be utilized to collimate the light prior to reaching the insertion site, such as described herein. In some cases, a seal may be placed around the proximal end 610 (such as a silicone skirt 416 discussed above in or around the indent shown at the proximal end 610) so that light is less likely to leak out of the exit opening at the proximal end 610.

The guide channel 602 may assist in guiding the needle and cannula and/or analyte sensor in directing or maintain a straight path for the entrance of the needle and cannula and/or analyte sensor into the patient’s skin. For example, the guide channel 602 can help to keep the needle and cannula and/or analyte sensor from twisting, turning, swiveling, etc., due to its shape. For example, as shown in FIGS. 6A and 6B, a shape of a guide channel 602 may be asymmetrical to limit rotation of an asymmetrical needle or other implantable device.

The overall cylindrical shape of the UV reflector 608 may include a diameter of approximately 1.50 mm - 4.50 mm. In some embodiments, the diameter of the UV reflector 608 may be approximately 3.50 mm. The overall height of the UV reflector may include a height approximately between 2 mm to 6 mm. In some embodiments, the overall height of the UV reflector may be approximately equal to 4 mm.

The UV reflector 608 may have one or more light channels that run widthwise through the reflector body, such that the light channel 604 connects the surface of the reflector body to the center of the reflector. For example, the one or more light channels 604 may be configured to receive and/or direct light from the light pipes at a roughly perpendicular angle to a centerline of the UV reflector 608. The one or more light channels 604 may be configured to couple to one or more light pipes of the system described herein. This may allow the light to collimate within the UV reflector 608. The light channel may include a diameter of approximately 0.15 mm to 0.3 mm. In some embodiments, the diameter of the light channel 604 may be approximately equal to 0.25 mm. The light channel 604 may be located approximately 2.86 mm above the distal end of the UV reflector 608. The location of the light channel 604 relative to the distal end of the UV reflector 608 may help to regulate or control sterilization of the insertion site and/or light intensity. For example, the location of the light channel 604 relative to the distal end of the UV reflector 608 may determine the distance the light may travel to exit the UV reflector and come in contact with the insertion site.

The UV reflector 608 may include a hollow body or an interior cavity 606 that may include a reflective surface aligning the inside of the hollow body or interior cavity 606. The reflective surface may include an aluminum reflective coating or surface that may help to regulate or control the sterilization of the insertion site. For example, the aluminum reflective surface may help to direct the light through the UV reflector. The aluminum reflective surface may extend from the distal end to a proximal end of the UV reflector 608 to a height of approximately 1.5 mm to 3.5 mm. In some embodiments, the height of the UV reflector 608 may be approximately equal to 2.86 mm.

An interior cavity of the UV reflector 608 may include shapes such as a tube 607 and/or angled surface 605. In some examples, the interior cavity 606 may be configured to connect to or receive light from one or more light channels 604. While a certain shape is shown in FIGS. 6A-6D and noted herein, such as a tube, other shapes of the cavity are also possible. The tube 607 may define or outline the inner cavity 606 of the UV reflector 608. The tube 607 may have a diameter of approximately 1.00 mm to 2.00 mm. In some embodiments, the tube 607 may have a diameter approximately equal to 1.20 mm. A diameter of an angled surface 605 of the UV reflector 608 may be approximately 1.5 mm to 2.5 mm. In some embodiments, the diameter may be approximately equal to 1.80 mm. The tube may include a reflective surface. For example, the tube 607 may include an aluminum reflective coating.

The angled surface 605 configured to reflect light from light channels 604 towards a centerline of the cavity 606. The angled surface 605 may have an angle of approximately 30-60 degrees. In some examples, the angled surface may be approximately 47.5 degrees with respect the light channel 604 and/or centerline axis of the cavity 606. The angled surface 605 may additionally be reflective so as to help ensure the light received into the UV reflector 608 is direct to the distal end of the UV reflector. In other words, the angled surface 605 may help to ensure the emitted light is directed to the insertion site. The angled surface 605 may be reflective as a result of an aluminum reflective coating which may or may not be the same as the coating of the rest of the cavity 606.

The UV reflector 608 may be press-fit into the top shell 500, as shown in 5A-5B. In some examples, press-fitting the UV reflector 608 into the top shell 500 may reduce errors caused by thermal expansion. Other methods of fitting the UV reflector 708 into the top shell 500 is also possible.

The guide channel 602 may allow for passage of the needle cannula or analyte sensor needle to the point of insertion into the skin. The guide channel 602 may control and/or limit the movement of the needle cannula or analyte sensor needle as it passes. Similarly, the guide channel 602 may minimize error in insertion of the needle cannula or analyte sensor needle. This may minimize any uncomfortableness of the patient and help to ensure the insertion is minimally invasive. The guide channel 602 may include a diameter of approximately 0.40 mm to 0.90 mm. In some embodiments, the guide channel 602 may have a diameter of approximately 0.70 mm.

The UV reflector 608 may be designed to allow for a portion of the reflector to be conical in shape, forming the angled surface 605. The angled surface 605 of the UV reflector 608 can help to ensure emitted light is directed to the point of insertion. The angled surface 605 of the UV reflector 608 may connect to the tube 607 to form the cavity 606. In this manner, the triangular shape 605a formed by the angled surface 605 may be include a reflective surface so that light may be received and reflected along the axis of the UV reflector 608. For example, the internal surface formed by the hypotenuse of the triangular shape 605a may be reflective to allow light to be received and reflected along the axis of the UV reflector 608. This may direct the light to the point of insertion.

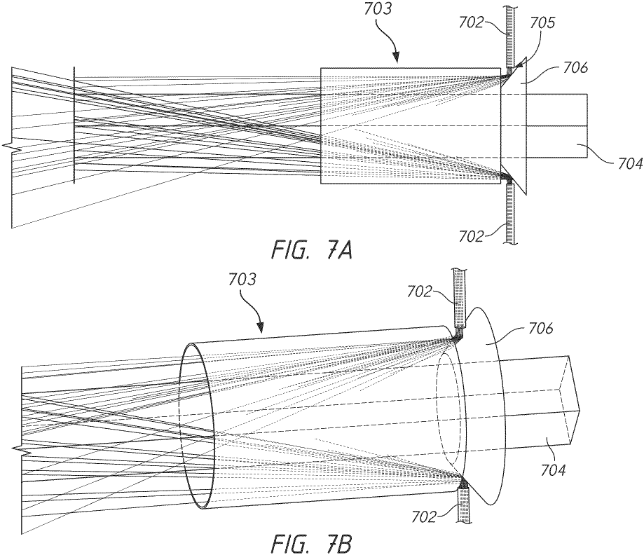

FIGS. 7A-7B illustrates an optical system. The optical system may include and outer tube 703 and an inner tube 706 of the UV reflector, a guide shape 704, and UV-C light 702. The UV-C light 702 may come from a lamp, laser, or the like. Such lamp or laser may include an optical fiber. The dimensions of the optical fiber may include approximately 100 µm Φ core and cladding approximately equal to 125 µm Φ. Specifically, the optical fiber may be solarization-resistant and operate within a wavelength of 180 nm-850 nm and obtain a numerical aperture of 0.22.

The light source may emit collimated UV-C light 702 that reflects from the inner tube 706 of the UV reflector. The outer tube 703 and the inner tube 706 are connected via an angle 705 that may range from 45-55 degrees. For example, the angle 705 may be equal to approximately 47.5 degrees. The angle 705 causes the UV-C light 702 to reflect internally down the length of the reflector. An interior surface of the reflector is illustrated in FIGS. 7A and 7B to illustrate how light may be reflected along the internal cavity of the UV reflector. Internal reflection of the UV-C light within the UV reflector may provide control of the light within its path to sterilization of the skin. One or more implantable components (represented by shape 704), which may include needles, cannulas, probes, or the like, which may pass through center of UV reflector. Furthermore, throughout the duration of its path, the UV-C light 702 may diffract at several different angles. Such diffraction may provide for sterilization down the length of guide shape 704 to the insertion point and surrounding skin in addition to sterilization of the implantable components.

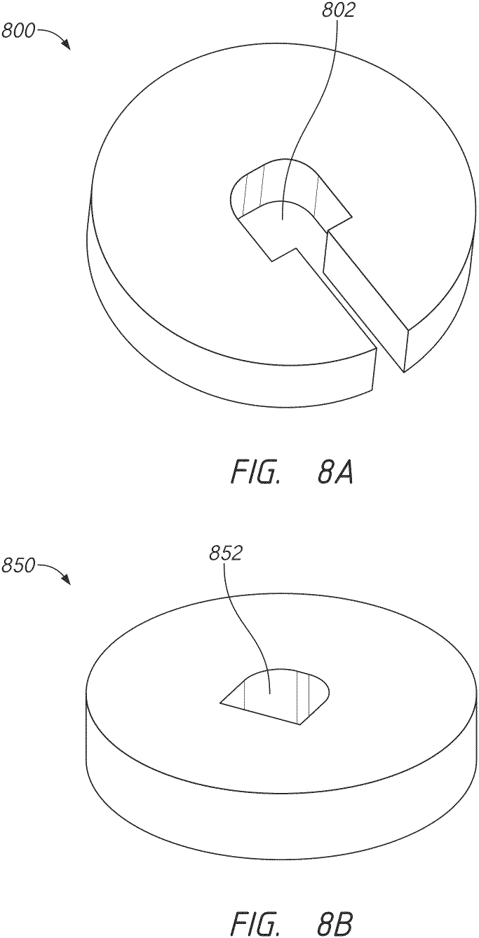

FIGS. 8A-8B illustrates an example of UV windows 800, 850. The UV windows may have a cylindrical shape. The UV windows 800, 850 may function as a diffuser of the light to the point of insertion. Diffusing the light may help to sterilize a greater area of the insertion site. In other words, diffusing the light may increase the area of the insertion site that is sterilized or exposed to the light. The UV windows 800, 850 may be flush against the skin to diffuse the received light into a larger spot size on the skin. The flushness of the UV windows 800, 850 can help to ensure that light directed to the point of insertion comes in contact with the insertion site. This can increase the degree of sterilization of the insertion site. The larger the spot size the received light creates may lead to a greater area of the insertion site that is sterilized. As described above, the light emitted by the UV source may be received by the UV reflectors, where the light may be collimated and reflected inward along the axis of the UV reflectors. The UV windows 800, 850 may receive the light from the UV reflectors to diffuse the received light on to skin to sterilize the point of insertion. The spot size of which the light creates may contribute to the frequency of re-sterilization. A greater diffusion of light may generally correlate with a decrease in the need for re-sterilization. This may affect the battery power and efficiency of the system overall. For example, the less frequent the insertion site may need to be re-sterilized, the more efficient the system may be and the less burdensome the use of the minimally invasive system for the user. The UV window 800, 850 may include a cut-out 802, 852 that allows for the needle cannula and analyte sensor needle to be received by the UV windows 800, 850. The cut-out 802, 852 can better control and/or direct the needle cannula and analyte sensor needle to the intended point of insertion. As shown in FIG. 8A, the cut-out 802 may extend the radius of the UV window 800.

### D. Light Source Conditioning

Conditioning of a light source used for sterilization can be critical to prevent harm to mammalian tissue. In some examples, conditioning of the light source used for sterilization may prevent harm to mammalian tissue Conditioning may include cleaning the UV light source by bandpass filtering the emitted UV light with diffraction gratings, interference, and/or absorptive-based filters. The bandwidth of the UV-C bandpass filter should be approximately 10-50 nm in width, such as approximately 25 nm. The selected band may be, for example, from approximately 200 to 225 nm or a range less than or greater than that range. If a lasing light source is used, a filter step may not be needed to further clean the light source.

If interference filters are used, prior to filtering, the system may first shape the emitted light with some form of collimation, such as a lens, polarizer, diffraction gratings, Narrow NA waveguide or fiber. Additionally or alternatively, the light source itself may be collimated, such as a light source that is lasing, thereby already having a tight beam to achieve adequate suppression. Beam collimation by shaping and/or from a collimated light source should not deviate by more than approximately 15 degrees from the normal angle to achieve the necessary light attenuation for safe cleaning of such a light source.

A fluence rate of UV light may be selected and/or determined based on the desired level of sterilization. A fluence rate of cleaned light of at least approximately 10, 20, and 40 mJ/cm² or (mW ∗ sec)/cm² may be applied to achieve sterilization of 1e-3, 1e-5, and 1e-6 Sterility Assurance Level (SAL) respectively. For example, a one second exposure to a 10 mW/cm^2 light source may be needed for sterilization for 1e-3 SAL. In another example, a one second exposure to a 20 mW/cm² light source may be needed for sterilization for 1e-5 SAL. In another example, a one second exposure to a 40 mW/cm² light source may be needed for sterilization for 1e-6 SAL.

An optional diffuser may be used to spread a more powerful light source to the desired fluence rate across the surface to be sterilized, such as the user’s skin and/or insertion site. A cannula of an insulin pump or analyte sensor part of an analyte monitor (such as a continuous glucose monitor or CGM) may be used as a light conductor and/or diffuser to spread light across the surface to be sterilized. The material of the diffuser may be selected to diffuse ultraviolet light in the selected range. For example, a diffuser may not function properly if the diffuser may be composed of plastic due to plastic’s high UV absorbance. In some examples, diffuser material may include ground glass or ground quartz windows. In some examples, widespread skin contact with ultraviolet light may be achieved by making an adhesive plate or base plate of a minimally invasive device a UV222 diffuser. Skin contact may be considered widespread if, for example, a spot size of emitted light is greater than or equal to 0.5 cm². As shown in FIG. 2 an adhesive layer 1168 of a minimally invasive implant may have a through hole to pass diffused light from a UV222 diffusing base plate 1106. In some examples, care may be needed to integrate a glass diffuser into a disposable plastic shell in a cost-effective manner while still maintaining an effective seal, such as an IP58 seal of the device.

### E. Modulation Determination

During use, re-exposure of the skin or other components of the system to ultraviolet light may be required to maintain a desired level of sterilization. For example, growth of bacteria commonly found on skin, such as ee-coli, may be rapid enough to pose a new risk of infection after a period of use of a minimally invasive implant. Using the example of ee-coli, response of an ee-coli bacterial colony perimeter to sterilization of a 5 um distance under ideal growth conditions would take roughly 12 hours to spread 700 um at a rate of approximately 58 um/hr. Extrapolating this rate of growth, gives roughly 5 minutes for the colony to reach the insertion point again at 5 [um] radius. However, any movement can easily shear the skin surface and help provide mobility to a colony back to the insertion site. It is for this reason, a much higher re-sterilization frequency may be recommended of 0.6 sec/min or a 1% Duty cycle roughly would be acceptable for a 10 mW/cm² source.

In some examples, larger light source diffusions pre-skin contact may further reduce the frequency. For example, if a 5.6 mm encroachment radium occurred, it may take approximately 10 hours for the bacteria perimeter to reach the insertion for a second time. Therefore, less frequent, 1 second UV exposure may be needed approximately every few hours. The surface that may be sterilized may include the entire underside of the device, which may be an area of 20-30 cm². Sterilization may also occur in a more focused area around the site of insertion, which may include an area of approximately 0.5 cm². The greater the spot size that the UV-C light may encroach, the less time may be needed to sterilize the area. For example, approximately a 1 second exposure over 3600 seconds may be needed.

Generally, power consumption may be considered. In some examples, if a light source is powerful enough to diffuse before skin contact to a fluence of 10 mW/cm² over 1 cm², a 10 mW light source may be used. This source may only need to be turned on for 1 second, once per hour to ensure 1e-3 SAL. In some examples, if a 1e-6 SAL is desired, the source may need to consume approximately four times more exposure or 4 seconds. Assuming a 50% conversion efficiency, a 10*(4/3600)*2 = 0.02 mW for 1 hour of sterilization may be needed. In some examples, if a fourteen-day device is used, a 0.02*14*24=6.72 mW hr of power may be needed. In some examples, if both a cannula and CGM insertion need separate coverage, a device may need 15 mWhr. The power consumption of the light source may range from 0.1 mW/hr to 2.4 mW/hr. This may depend on the sterilization level, which may range from 1e-3 SAL to 1e-6 SAL for a sterilization area ranging between 0.5 cm² to 30 cm².

### F. Terminology

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps but may include additional steps. When used in the context of a device, the term “comprising” means that the device includes at least the recited features or components but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid-state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A minimally invasive implant worn flush against a patient’s skin comprising:
an implantable component configured to at least partially implant into the patient’s skin for a period of time of use of the minimally invasive implant;
a UV light emitter configured to emit UV-C light;
at least one UV reflector configured to receive light from the UV light emitter, the at least one UV reflector comprising:
an interior cavity comprising:
an implant receiving portion running lengthwise along the at least one UV reflector and configured to receive at least some of the implantable component; and
an angled surface configured to receive UV-C light from at least one light channel perpendicular to a centerline of the interior cavity and to direct the received UV-C light towards an exit opening of the interior cavity such that the implantable component is sterilized by the UV-C light, wherein the at least one light channel is configured to receive the UV-C light from the UV light emitter; and
a UV window positioned between the at least one UV reflector and the patient’s skin such that diverted UV-C light shines through the UV window to sterilize the patient’s skin, the UV window comprising an opening configured to allow the implantable component to at least partially pass through the UV window to implant in the patient’s skin.

2. The minimally invasive implant of claim 1, wherein the interior cavity is reflective to UV-C light and able to receive UV-C light to reflect the UV-C light within the at least one UV reflector towards a proximal exit opening of the UV reflector.

3. The minimally invasive implant of claim 1, wherein the UV light emitter is connected to the at least one UV reflector via at least one light pipe.

4. The minimally invasive implant of claim 3, wherein the UV light emitter includes a first UV chip configured to emit light to the UV reflector and a second UV chip configured to emit light to a second UV reflector.

5. The minimally invasive implant of claim 4, wherein the UV reflector receives a first needle and an analyte sensor, and the second UV reflector receives a second needle and a cannula.

6. The minimally invasive implant of claim 1, wherein the angled surface comprises an angle of approximately 47.5 degrees.

7. The minimally invasive implant of claim 1, wherein the at least one UV reflector includes a diameter of approximately 3.50 mm and a height of approximately 3 mm.

8. The minimally invasive implant of claim 1, wherein the at least one UV reflector further comprises a tube extending towards an exit opening of the cavity.

9. The minimally invasive implant of claim 1, wherein the at least one UV reflector couples to a top shell of a disease management device.

10. The minimally invasive implant of claim 1, wherein the UV window couples to a lower shell of a disease management device.

11. The minimally invasive implant of claim 1, wherein the UV-C light undergoes a filtration process prior to sterilizing the patient’s skin.

12. The minimally invasive implant of claim 1, wherein the UV light emitter is further configured to emit light periodically.

13. The minimally invasive implant of claim 12, wherein the UV light emitter is further configured to provide a 1 second exposure over 3600 seconds.

14. The minimally invasive implant of claim 1, wherein a light channel is positioned to connect an outer surface of the at least one UV reflector to the angled portion of the at least one UV reflector.

15. A method to sterilize minimally invasive device and insertion site of a patient, the method comprising:
emitting UV-C light from a UV emitter periodically to be received by at least one UV reflector within a disease management system; and
reflecting UV-C light within the at least one UV reflector to sterilize at least a portion of the disease management system and insertion site of the patient at a predetermined frequency.

16. The method of claim 15, wherein the predetermined frequency is based on an area of the insertion site.

17. The method of claim 15, wherein the predetermined frequency is based on a degree of risk for infection at the insertion site.

18. A method to sterilize an insertion site of a patient, the method comprising:
inserting a needle of a disease management system into the insertion site of the patient;
collimating UV light to enter at least one UV reflector located within an UV sterilization device;
reflecting UV light to direct the UV light inward toward a center axis of the at least one UV reflector, wherein the needle is positioned within with the center axis of the at least one UV reflector;
diffusing the UV light through a UV window upon the UV light exiting the at least one UV reflector, wherein the UV window is configured to allow the needle to pass through the UV window to the insertion site of the patient; and
sterilizing the insertion site of the patient.

19. The method of claim 18 further comprising conditioning a light source prior to collimating UV light.

20. The method of claim 19 wherein conditioning the light source includes bandpass filtering emitted UV light.

21. The method of claim 18, wherein the UV window is flush against the patient.

22. The method of claim 18 further comprising re-sterilizing the insertion site of the patient based on a spot size of the UV light.
